(19) United States
(12) Patent Application Publication
(10) Pub. No.: US 2025/0010005 A1
(43) Pub. Date: Jan. 9, 2025

(54) CARBON DIOXIDE AND OTHER GAS COMPENSATION DEVICE AND SYSTEM CONNECTED TO A UNIVERSAL VENTILATOR

(71) Applicant: GUANGZHOU LANDSWICK MEDICAL TECHNOLOGIES LTD., Guangzhou (CN)

(72) Inventors: Hui DONG, Guangzhou (CN); Caixin SUN, Guangzhou (CN)

(21) Appl. No.: 18/648,488

(22) Filed: Apr. 29, 2024

Related U.S. Application Data
(63) Continuation of application No. PCT/CN2024/074141, filed on Jan. 26, 2024.

(30) Foreign Application Priority Data
Jul. 3, 2023 (CN) 2023108048008

(51) Int. Cl. A61M 16/00; A61M 16/04; A61M 16/06; A61M 16/10; A61M 16/20

(52) U.S. Cl. CPC A61M 16/024; A61M 16/0003; A61M 16/0066; A61M 16/04; A61M 16/06; A61M 16/1075; A61M 16/203; A61M 2016/0027; A61M 2016/0039; A61M 2016/103; A61M 2202/0007; A61M 2202/0225; A61M 2205/103; A61M 2205/3303; A61M 2205/3327; A61M 2205/3334; A61M 2205/3344; A61M 2205/3365; A61M 2205/3653; A61M 2205/502; A61M 2205/75; A61M 2230/43

(57) ABSTRACT

A CO₂ and other gas compensation device includes a device body containing a respiratory gas CO₂ compensation device. Inside the respiratory gas CO₂ compensation device, an airway is set up. An output end of the airway is connected to a respiratory parameter detection module, which is connected to the respiratory terminal. Inside the respiratory terminal, a proximal pressure sensor and an end-tidal CO₂ sensor are installed. The proximal pressure sensor and the end-tidal CO₂ sensor are electrically connected to the respiratory gas CO₂ compensation device. CO₂ is compensated into the respiratory terminal through the respiratory gas CO₂ compensation device. The respiratory parameter detection module can monitor parameters of incoming CO₂. The respiratory gas CO₂ compensation device, in coordination with the respiratory parameter detection module, achieves precise monitoring and adjustment of the incoming CO₂, thereby accurately controlling the amount of CO₂ compensation required by the patient and improving the treatment effect.

### TECHNICAL FIELD

The disclosure relates to the medical technology field, particularly to a carbon dioxide (CO₂) and other gas compensation device and system connected to a universal ventilator.

### BACKGROUND

Traditional ventilators do not have CO₂ compensation functionality. When used, if the patient's end-tidal carbon dioxide (EtCO₂) is low and the carbon dioxide partial pressure is low, it can cause the patient to suffer from respiratory alkalosis; if the end-tidal carbon dioxide is high and the carbon dioxide partial pressure is high, it indicates CO₂ retention, which can cause the patient to suffer from respiratory acidosis.

Currently, for low end-tidal carbon dioxide partial pressure (respiratory alkalosis), a bag or collection device is generally used to cover the patient's mouth and nose, allowing the patient to re-inhale the exhaled carbon dioxide. For example, the patent with the publication number “CN103536995B” reveals an oxygen mask, including a mask body and a gas storage device. A first one-way valve is set between the mask body and the gas storage device, and a first air hole and a second air hole are set between the first one-way valve and the gas storage device. The mask body and the gas storage device are connected through a pipeline structure, which includes interconnected first and second pipelines. The first pipeline has a first air hole and a third air hole on its wall, and the second pipeline has a fourth air hole. The fourth air hole is connected to the second air hole. This structure allows part of the exhaled carbon dioxide to remain in the second pipeline during exhalation. During inhalation, this part of carbon dioxide is inhaled into the first pipeline and mixed with oxygen from the storage gas device, thus avoiding discomfort caused by excessive carbon dioxide expulsion due to high oxygen concentration inhalation.

However, the above-mentioned oxygen mask cannot precisely control the amount of carbon dioxide gas needed by the patient, nor can it precisely monitor and adjust the incoming carbon dioxide, failing to reflect the patient's end-tidal carbon dioxide situation, thus affecting the treatment effect of the ventilator.

### SUMMARY

This disclosure provides a CO₂ and other gas compensation device and system connected to a universal ventilator, aiming to solve the technical problem of current devices not being able to precisely control the amount of carbon dioxide gas needed by the patient, nor precisely monitor and adjust the incoming carbon dioxide, failing to reflect the patient's end-tidal carbon dioxide situation, thus affecting the treatment effect of the ventilator.

To solve the above technical problem, this disclosure discloses a CO₂ and other gas compensation device, configured to be connected to a universal ventilator, including: the device body, which contains a respiratory gas CO₂ compensation device. Inside the respiratory gas CO₂ compensation device, an airway is set up. The output end of the airway is connected to a respiratory parameter detection module, which is connected to the respiratory terminal. Inside the respiratory terminal, a proximal pressure sensor and an end-tidal carbon dioxide sensor are installed. The proximal pressure sensor is used to detect the gas pressure inside the respiratory terminal, and the end-tidal carbon dioxide sensor is used to detect the end-tidal carbon dioxide value inside the respiratory terminal. The proximal pressure sensor and the end-tidal carbon dioxide sensor are electrically connected to the respiratory gas CO₂ compensation device, individually. The respiratory gas CO₂ compensation device is configured to deliver carbon dioxide into the respiratory terminal based on the detection values of the proximal pressure sensor and the end-tidal carbon dioxide sensor through the respiratory parameter detection module.

Preferably, the respiratory terminal is either a mask or a breathing tube intubation.

Preferably, an intake filter is set near the input end of the airway, and an exhaust filter is set near the output end of the airway.

Preferably, the device body also includes an adjustment control system (i.e., regulation control system), which includes a control unit, an execution unit, and a detection unit. The control unit includes a controller, which is set inside the respiratory gas CO₂ compensation device. The controller is electrically connected to the proximal pressure sensor through the first cable and to the end-tidal carbon dioxide sensor through the second cable. The execution unit includes a solenoid valve and a carbon dioxide regulation proportional valve, both set on the airway. The solenoid valve and the carbon dioxide regulation proportional valve are sequentially set between the intake filter and the exhaust filter and are electrically connected to the controller, individually. The detection unit includes an intake pressure sensor, a carbon dioxide flowmeter, and a carbon dioxide supply pressure sensor, sequentially set on the airway. The intake pressure sensor, carbon dioxide flowmeter, and carbon dioxide supply pressure sensor are electrically connected to the controller, individually. The intake pressure sensor is located between the intake filter and the solenoid valve, the carbon dioxide flowmeter is located between the carbon dioxide regulation proportional valve and the exhaust filter, and the carbon dioxide supply pressure sensor is located between the carbon dioxide flowmeter and the exhaust filter.

Preferably, the respiratory parameter detection module includes a compensation mixing tube. The compensation mixing tube uses a three-way tube. The first end of the compensation mixing tube is used for the airflow from the ventilator. The second end of the compensation mixing tube is connected to the output end of the airway through a connecting tube. A one-way valve is set inside the second end of the compensation mixing tube. The third end of the compensation mixing tube is connected to the interior of the respiratory terminal through a gas supply circuit.

Preferably, the detection unit also includes a flowmeter and a carbon dioxide concentration sensor. The flowmeter is set near the first end of the compensation mixing tube, and the carbon dioxide concentration sensor is set near the third end of the compensation mixing tube. The flowmeter is electrically connected to the controller through the third cable, and the carbon dioxide concentration sensor is electrically connected to the controller through the fourth cable.

Preferably, a supporting plate is set outside the airway. Several bolt mounting holes are set on the supporting plate. An electric heating plate is set inside the airway, located between the intake filter and the intake pressure sensor. The electric heating plate is electrically connected to the controller and is coaxially set inside the airway. Several through holes are opened on the electric heating plate. A fixed shaft is set in the center of the electric heating plate. Near the fixed shaft side of the electric heating plate, a fixed ring and a rotating ring are sequentially set. The outer circumference of the fixed ring is fixedly connected to the inner wall of the airway. A first convex ring is set on the side of the fixed ring facing away from the electric heating plate. The first convex ring is coaxially set with the fixed ring. The outer circumference of the rotating ring is rotatably connected to the inner wall of the airway. A second convex ring is set on the side of the rotating ring near the fixed ring. The inner side wall of the second convex ring is rotatably connected to the outer side wall of the first convex ring. Several baffles are set around the outer circumference of the fixed shaft. The baffles are made of flexible sheet material. Several baffles are arranged in a circular array around the center of the fixed shaft. One end of the baffle is connected to the outer circle of the fixed shaft, and the end of the baffle facing away from the fixed shaft is connected to the inner circumferential surface of the fixed ring on the side near the fixed ring and to the inner circumferential surface of the rotating ring on the side near the rotating ring. A driving component is set outside the airway. The driving component is used to drive the rotation of the rotating ring.

Preferably, the driving component includes a drive rod and an electric telescopic rod. One end of the drive rod penetrates the side wall of the airway and is connected to the outer wall of the rotating ring. A long slide slot (i.e., slide slot) compatible with the drive rod is set on the airway. The drive rod is slidably connected to the long slide slot. A strip groove is set on the end of the drive rod facing away from the rotating ring. The electric telescopic rod is set on the side wall of the supporting plate. The output end of the electric telescopic rod passes through the strip groove and sets a connecting rod. One end of the connecting rod is hingedly connected to the output end of the electric telescopic rod, and the other end of the connecting rod is hingedly connected to the inner wall of the upper end of the strip groove.

Preferably, several arc holes are opened on the rotating ring. Several arc holes are arranged in a circular array around the center of the rotating ring. A guide column is slidably set inside each arc hole. One end of the guide column is fixedly connected to the side of the fixed ring near the rotating ring, and the other end of the guide column extends outside the arc hole and sets a limit block.

This disclosure also provides a CO₂ and other gas compensation system connected to a universal ventilator, including the aforementioned CO₂ and other gas compensation device connected to a universal ventilator, as well as a ventilator and a carbon dioxide gas source. The carbon dioxide gas source is connected to the input end of the airway of the respiratory gas CO₂ compensation device through an intake pipe. The ventilator is connected to the compensation mixing tube through a respiratory circuit.

The technical solution of this disclosure has the following advantages: This disclosure provides a CO₂ and other gas compensation device and system connected to a universal ventilator, related to the medical technology field. It includes the device body, which contains a respiratory gas CO₂ compensation device. Inside the respiratory gas CO₂ compensation device, an airway is set up. The output end of the airway is connected to a respiratory parameter detection module, which is connected to the respiratory terminal. Inside the respiratory terminal, a proximal pressure sensor and an end-tidal carbon dioxide sensor are installed. The proximal pressure sensor is used to detect the gas pressure inside the respiratory terminal, and the end-tidal carbon dioxide sensor is used to detect the end-tidal carbon dioxide value inside the respiratory terminal. The proximal pressure sensor and the end-tidal carbon dioxide sensor are electrically connected to the respiratory gas CO₂ compensation device, respectively. The respiratory gas CO₂ compensation device delivers carbon dioxide into the respiratory terminal based on the detection values of the proximal pressure sensor and the end-tidal carbon dioxide sensor through the respiratory parameter detection module. In this disclosure, the end-tidal carbon dioxide sensor can detect the end-tidal carbon dioxide value inside the respiratory terminal, thereby reflecting the patient's end-tidal carbon dioxide situation. The respiratory gas CO₂ compensation device can compensate carbon dioxide gas into the respiratory terminal. The respiratory parameter detection module can monitor the parameters of the incoming carbon dioxide. The respiratory gas CO₂ compensation device, in coordination with the respiratory parameter detection module, achieves precise monitoring and adjustment of the incoming carbon dioxide, thereby accurately controlling the amount of carbon dioxide compensation required by the patient and improving the treatment effect of the ventilator.

Other features and advantages of this disclosure will be described in the following description, and, in part, will become apparent from the description, or may be learned by the practice of this disclosure. The objectives and other advantages of this disclosure may be realized and obtained through the devices particularly pointed out in the written description and claims as well as the appended drawings.

The following will further detail the technical solution of this disclosure through the drawings and embodiments.

### BRIEF DESCRIPTION OF THE DRAWINGS

The drawings are intended to provide further understanding of the disclosure and constitute a part of this description, used together with the embodiments of the disclosure to explain the disclosure, without limiting the disclosure. In the drawings:

FIG. 1 is a schematic diagram of the overall structure of a CO₂ and other gas compensation device connected to a universal ventilator according to this disclosure;

FIG. 2 is a schematic diagram of the internal structure of the compensation mixing tube in this disclosure;

FIG. 3 is a schematic diagram of the internal structure of the respiratory gas CO₂ compensation device in this disclosure;

FIG. 4 is a schematic diagram of the adjustment control system in this disclosure;

FIG. 5 is a workflow diagram of a CO₂ and other gas compensation system connected to a universal ventilator according to this disclosure;

FIG. 6 is a schematic diagram of the internal structure of the airway in this disclosure;

FIG. 7 is a schematic diagram of the structure of the electric heating plate in this disclosure;

FIG. 8 is an enlarged diagram of the structure at location A in FIG. 7 of this disclosure;

FIG. 9 is a partial sectional view of the electric heating plate in this disclosure.

### LIST OF REFERENCE NUMERALS

1—carbon dioxide gas source; 101—intake pipe; 2—ventilator; 3—respiratory circuit; 4—respiratory parameter detection module; 41—flowmeter; 411—third cable; 412—fourth cable; 42—compensation mixing tube; 421—connecting tube; 422—one-way valve; 43—carbon dioxide concentration sensor; 441—intake filter; 442—intake pressure sensor; 443—solenoid valve; 444—carbon dioxide regulation proportional valve; 445—carbon dioxide flowmeter; 446—carbon dioxide supply pressure sensor; 447—exhaust filter; 45—controller; 5—respiratory terminal; 6—patient; 7—proximal pressure sensor; 71—first cable; 8—end-tidal carbon dioxide sensor; 81—second cable; 9—respiratory gas CO₂ compensation device; 10—airway; 1001—supporting plate; 10011—bolt mounting hole; 10012—slide slot; 1002—electric heating plate; 1003—through hole; 1004—fixed shaft; 1005—fixed ring; 1006—rotating ring; 1007—first convex ring; 1008—second convex ring; 1009—baffle; 1010—drive rod; 1011—electric telescopic rod; 1012—strip groove; 1013—connecting rod; 1014—arc hole; 1015—guide column; 1016—limit block; 11—gas supply circuit.

### DETAILED DESCRIPTION OF EMBODIMENTS

The following describes preferred embodiments of this disclosure in conjunction with the drawings. It should be understood that the preferred embodiments described herein are only for illustrating and explaining this disclosure and are not intended to limit this disclosure.

Additionally, descriptions such as “first,” “second,” etc., in this disclosure are only for descriptive purposes and are not meant to indicate or imply relative importance or the implicit indication of the number of technical features indicated. Thus, features defining “first,” “second,” can include one or more of such features. Furthermore, the technical solutions and technical features among various embodiments can be combined, but they must be based on the realization by those skilled in the art, and when the combination of technical solutions appears contradictory or unachievable, it should be considered that the combination of these technical solutions does not exist and is not within the scope of protection of this disclosure.

### Embodiment 1

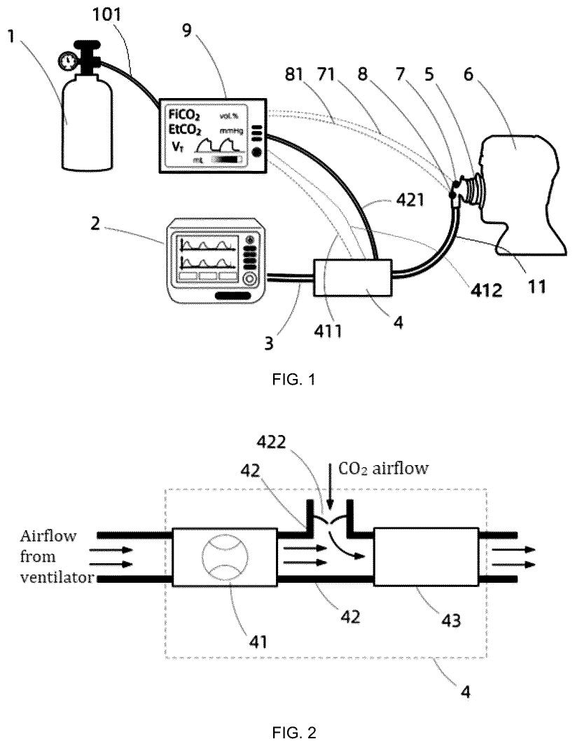

An embodiment of this disclosure provides a CO₂ and other gas compensation device connected to a universal ventilator, as shown in FIG. 1, including: the device body, which contains a respiratory gas CO₂ compensation device 9. Inside the respiratory gas CO₂ compensation device 9, an airway 10 is set up. The output end of the airway 10 is connected to a respiratory parameter detection module 4, which is connected to the respiratory terminal 5. Inside the respiratory terminal 5, a proximal pressure sensor 7 and an end-tidal carbon dioxide sensor 8 are installed. The proximal pressure sensor 7 is used to detect the gas pressure inside the respiratory terminal 5, and the end-tidal carbon dioxide sensor 8 is used to detect the end-tidal carbon dioxide value inside the respiratory terminal 5. The proximal pressure sensor 7 and the end-tidal carbon dioxide sensor 8 are electrically connected to the respiratory gas CO₂ compensation device 9, individually. The respiratory gas CO₂ compensation device 9 delivers carbon dioxide into the respiratory terminal 5 based on the detection values of the proximal pressure sensor 7 and the end-tidal carbon dioxide sensor 8 through the respiratory parameter detection module 4.

The respiratory terminal 5 can be either a mask or a breathing tube intubation.

The working principle and beneficial effects of the above technical solution are: The device body includes a respiratory gas CO₂ compensation device 9, a respiratory parameter detection module 4, and a respiratory terminal 5. The respiratory terminal 5 can be either a mask or a breathing tube intubation for the patient 6 to use for breathing. The respiratory gas CO₂ compensation device 9 has an airway 10 set up inside, with the input end of the airway 10 able to connect to a gas source, including a carbon dioxide gas source 1 or other active gases needed by the patient 6. To prevent the patient 6 from experiencing respiratory alkalosis when using the ventilator 2, it is necessary to provide carbon dioxide to the patient 6 through the respiratory terminal 5. Taking carbon dioxide gas as an example, one end of the airway 10 is connected to the carbon dioxide gas source 1, and the other end of the airway 10 is connected to the respiratory parameter detection module 4. The output end of the respiratory parameter detection module 4 is connected to the respiratory terminal 5. At this point, starting the respiratory gas CO₂ compensation device 9 can allow carbon dioxide to enter the airway 10 from the input end, then flow into the respiratory parameter detection module 4 along the airway 10. The respiratory parameter detection module 4 can monitor the parameters of the carbon dioxide entering the airway 10. Then, the carbon dioxide gas flows into the respiratory terminal 5 through the output end of the respiratory parameter detection module 4, thus providing carbon dioxide to the patient 6 and avoiding respiratory alkalosis in the patient 6. The proximal pressure sensor 7 and the end-tidal carbon dioxide sensor 8 are set inside the respiratory terminal 5. The proximal pressure sensor 7 can detect the gas pressure inside the respiratory terminal 5. The end-tidal carbon dioxide sensor 8 can detect the end-tidal carbon dioxide value inside the respiratory terminal 5, thereby reflecting the patient 6's end-tidal carbon dioxide situation. The respiratory gas CO₂ compensation device 9 can compensate carbon dioxide gas or other active gases into the respiratory terminal 5. The respiratory parameter detection module 4 can monitor the parameters of the incoming carbon dioxide. The respiratory gas CO₂ compensation device 9, in coordination with the respiratory parameter detection module 4, achieves precise monitoring and adjustment of the incoming carbon dioxide, thereby accurately controlling the amount of carbon dioxide gas needed by the patient 6 and improving the treatment effect of the ventilator 2.

### Embodiment 2

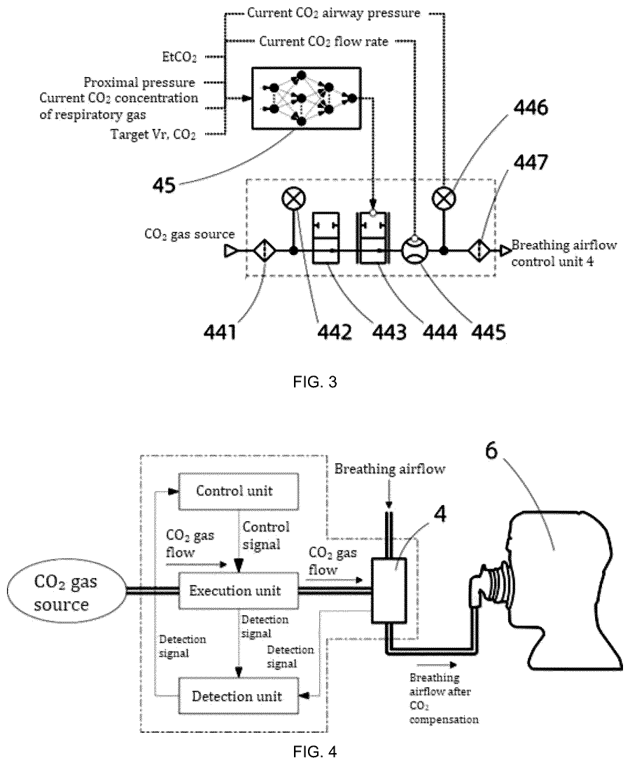

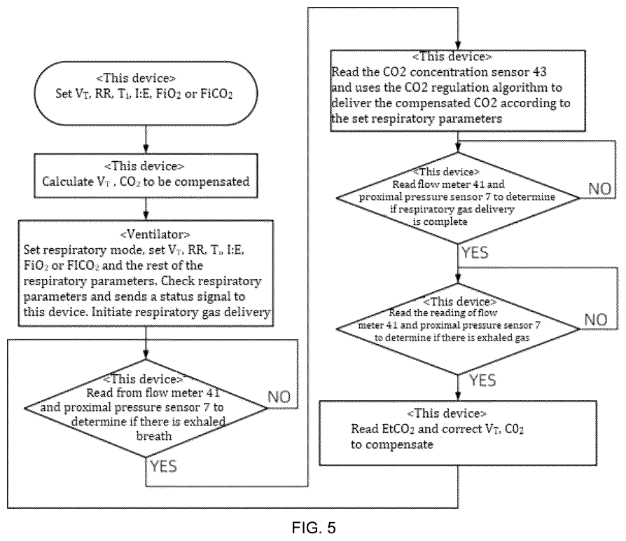

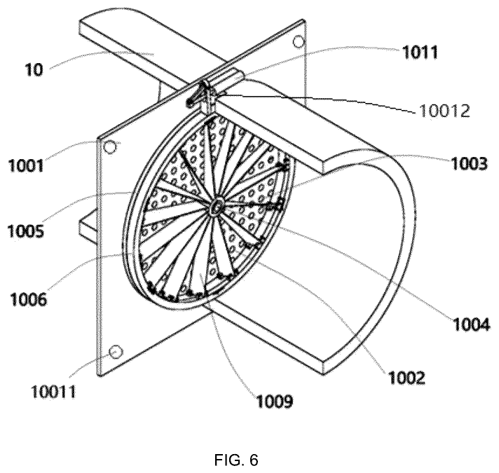

Based on Embodiment 1, as shown in FIGS. 1-5, the airway 10 has an intake filter 441 set near the input end and an exhaust filter 447 set near the output end.

The device body also includes an adjustment control system, which includes a control unit, an execution unit, and a detection unit. The control unit includes a controller 45, which is set inside the respiratory gas CO₂ compensation device 9. The controller 45 is electrically connected to the proximal pressure sensor 7 through the first cable 71 and to the end-tidal carbon dioxide sensor 8 through the second cable 81. The execution unit includes a solenoid valve 443 and a carbon dioxide regulation proportional valve 444, both set on the airway 10. The solenoid valve 443 and the carbon dioxide regulation proportional valve 444 are sequentially set between the intake filter 441 and the exhaust filter 447 and are electrically connected to the controller 45, individually. The detection unit includes an intake pressure sensor 442, a carbon dioxide flowmeter 445, and a carbon dioxide supply pressure sensor 446, sequentially set on the airway 10. The intake pressure sensor 442, carbon dioxide flowmeter 445, and carbon dioxide supply pressure sensor 446 are electrically connected to the controller 45, individually. The intake pressure sensor 442 is located between the intake filter 441 and the solenoid valve 443, the carbon dioxide flowmeter 445 is located between the carbon dioxide regulation proportional valve 444 and the exhaust filter 447, and the carbon dioxide supply pressure sensor 446 is located between the carbon dioxide flowmeter 445 and the exhaust filter 447.

The respiratory parameter detection module 4 includes a compensation mixing tube 42. The compensation mixing tube 42 uses a three-way tube. The first end of the compensation mixing tube 42 is used for the airflow from the ventilator 2. The second end of the compensation mixing tube 42 is connected to the output end of the airway 10 through a connecting tube 421. A one-way valve 422 is set inside the second end of the compensation mixing tube 42. The third end of the compensation mixing tube 42 is connected to the interior of the respiratory terminal 5 through a gas supply circuit 11.

The detection unit also includes a flowmeter 41 and a carbon dioxide concentration sensor 43. The flowmeter 41 is set near the first end of the compensation mixing tube 42, and the carbon dioxide concentration sensor 43 is set near the third end of the compensation mixing tube 42. The flowmeter 41 is electrically connected to the controller 45 through the third cable 411, and the carbon dioxide concentration sensor 43 is electrically connected to the controller 45 through the fourth cable 412.

The working principle and beneficial effects of the above technical solution are: The device body also includes an adjustment control system, which includes a control unit, an execution unit, and a detection unit. The control unit can send control signals to the execution unit. After receiving the control signals, the execution unit can allow the carbon dioxide gas flow from the carbon dioxide gas source 1 to enter the airway 10 and pass through the airway 10 to enter the compensation mixing tube 42 through its second end. The carbon dioxide entering the compensation mixing tube 42 mixes with the breathing airflow, thus achieving carbon dioxide compensation. The compensated breathing airflow then flows out from the third end into the respiratory terminal 5 for the patient 6 to breathe. The detection unit can obtain detection signals when carbon dioxide passes through the execution unit and the respiratory parameter detection module 4, and sends these detection signals to the control unit. The control unit then controls the execution unit based on the detection signals. Specifically, the control unit includes a controller 45 set inside the respiratory gas CO₂ compensation device 9. The execution unit includes a solenoid valve 443 and a carbon dioxide regulation proportional valve 444 set on the airway 10. The opening and closing of the solenoid valve 443 can control the connection and disconnection of the airway 10. The carbon dioxide regulation proportional valve 444 can adjust the pressure and flow of the carbon dioxide gas flow inside the airway 10. The detection unit includes an intake pressure sensor 442, a carbon dioxide flowmeter 445, a carbon dioxide supply pressure sensor 446 set on the airway 10, and a flowmeter 41 and a carbon dioxide concentration sensor 43 set inside the compensation mixing tube 42. When the respiratory terminal 5 needs carbon dioxide compensation, the controller 45 starts the solenoid valve 443 to connect the airway 10. The carbon dioxide gas flow provided by the carbon dioxide gas source 1 enters the airway 10 after passing through the intake filter 441. The intake pressure sensor 442 can detect the intake pressure of the carbon dioxide gas flow entering the airway 10. The carbon dioxide flowmeter 445 can detect the flow of the carbon dioxide gas flow inside the airway 10. The carbon dioxide supply pressure sensor 446 can detect the pressure of the carbon dioxide gas flow adjusted by the carbon dioxide regulation proportional valve 444. The adjusted carbon dioxide gas flow then passes through the exhaust filter 447 and enters the compensation mixing tube 42 through the connecting tube 421. A one-way valve 422 is set at the second end of the compensation mixing tube 42. The one-way valve 422 prevents the breathing airflow from the ventilator 2 from flowing into the connecting tube 421 through the second end. The carbon dioxide gas flow entering the compensation mixing tube 42 can mix with the breathing airflow provided by the ventilator 2, compensating for the carbon dioxide that is less in the original breathing airflow. The flowmeter 41 can detect the gas flow inside the compensation mixing tube 42. The carbon dioxide concentration sensor 43 can detect the carbon dioxide concentration of the compensated breathing airflow inside the compensation mixing tube 42 and send the detection results to the controller 45. Specifically, before use, the respiratory gas CO₂ compensation device 9 is set. The respiratory gas CO₂ compensation device 9 has a display screen that can display respiratory parameters such as VT, RR, Ti, I:E, FiO₂, FiCO₂, etc. VT represents tidal volume, RR represents ventilation frequency, Ti represents inhalation time, I:E represents the inhalation to exhalation ratio, FiO₂ represents the fraction of oxygen concentration in the inhaled gas, and FiCO₂ represents the fraction of carbon dioxide concentration in the inhaled gas. The display screen allows for a direct observation of the above parameters. The following is the working process of the device body, with the respiratory gas CO₂ compensation device 9 abbreviated as this device. First, set VT, RR, Ti, I:E, FiO₂, or FiCO₂ on this device. This device calculates the volume of carbon dioxide needed for compensation. During calculation, the volume of carbon dioxide in the exhaled gas can be obtained through the detection value of the end-tidal carbon dioxide sensor 8. Then, the volume of carbon dioxide needed for compensation can be obtained by subtracting the volume of carbon dioxide in the exhaled gas from the set volume of carbon dioxide. The set volume of carbon dioxide needs to be set according to the needs of different patients 6. Next, set the breathing mode, VT, RR, Ti, I:E, FiO₂, or FiCO₂, and other respiratory parameters on the ventilator 2. Check the respiratory parameters and send a status signal to this device. Start the delivery of respiratory gas. Then, this device reads the values of the flowmeter 41 and the proximal pressure sensor 7 to determine whether there is delivery of respiratory gas. If it is determined that there is no delivery of respiratory gas, repeat reading the values. If it is determined that there is delivery of respiratory gas, this device delivers the compensated carbon dioxide according to the set respiratory parameters based on the detection value of the carbon dioxide concentration sensor 43, using a carbon dioxide adjustment algorithm. The carbon dioxide adjustment algorithm requires that the sum of the compensated carbon dioxide concentration and the initial detection concentration of the carbon dioxide concentration sensor 43 be within the target carbon dioxide concentration range required in the patient's 6 respiratory airflow. The target carbon dioxide concentration range is set according to the needs of different patients 6. Then, this device reads the values of the flowmeter 41 and the proximal pressure sensor 7 to determine whether the delivery of respiratory gas has ended. If it is determined that the delivery has not ended, repeat reading the values. If it is determined that the delivery of respiratory gas has ended, this device reads the values of the flowmeter 41 and the proximal pressure sensor 7 to determine whether there is exhaled gas. If it is determined that there is no exhaled gas, repeat reading the values. If it is determined that there is exhaled gas, this device reads the end-tidal carbon dioxide value detected by the end-tidal carbon dioxide sensor 8 and corrects the volume of carbon dioxide needed for compensation based on the volume of carbon dioxide in the exhaled gas. This achieves precise control of the volume of carbon dioxide gas, as the disclosure automatically adjusts the volume and flow of carbon dioxide gas entering the universal ventilator 2 pipeline based on the gas source, feedback, monitoring, and sampling of the patient's 6 end-tidal carbon dioxide detection information, enhancing the automation, intelligence, and controllability of the device. Through high sensitivity CO₂ monitoring and adjustment control systems, the disclosure provides high precision carbon dioxide gas concentration needed by the patient 6, achieving effective treatment and medical purposes, and improving treatment outcomes.

### Embodiment 3

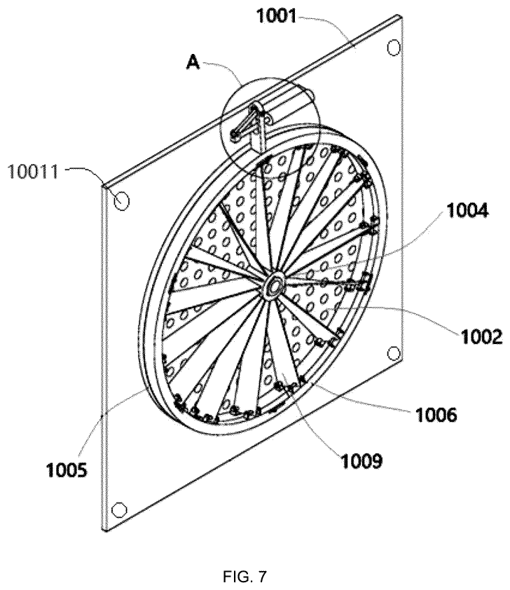

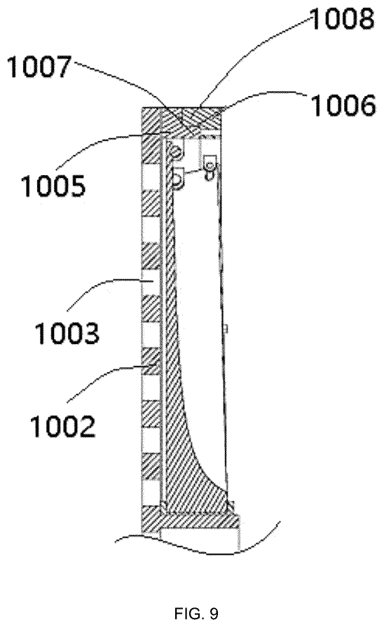

On the basis of Embodiment 2, as shown in FIGS. 6-9, a supporting plate 1001 is arranged outside the airway 10, and several bolt mounting holes 10011 are set on the supporting plate 1001. Inside the airway 10, an electric heating plate 1002 is arranged between the intake filter 441 and the intake pressure sensor 442. The electric heating plate 1002 is electrically connected to the controller 45 and is coaxially arranged inside the airway 10. The electric heating plate 1002 has several through holes 1003. In the center of the electric heating plate 1002, a fixed shaft 1004 is set. Near the fixed shaft 1004, a fixed ring 1005 and a rotating ring 1006 are arranged in sequence. The outer circumference of the fixed ring 1005 is fixedly connected to the inner wall of the airway 10. On the side of the fixed ring 1005 facing away from the electric heating plate 1002, a first convex ring 1007 is set, which is coaxially arranged with the fixed ring 1005. The outer circumference of the rotating ring 1006 is rotatably connected to the inner wall of the airway 10. On the side of the rotating ring 1006 close to the fixed ring 1005, a second convex ring 1008 is set, whose inner wall is rotatably connected to the outer wall of the first convex ring 1007. Several baffles 1009 are arranged around the outer circumference of the fixed shaft 1004. The baffles 1009 are made of flexible sheet material. Several baffles 1009 are arranged in a circular array around the center of the fixed shaft 1004. One end of the baffle 1009 is connected to the outer circle of the fixed shaft 1004, and the end of the baffle 1009 facing away from the fixed shaft 1004 is close to the side of the fixed ring 1005 and connected to the inner circumferential surface of the fixed ring 1005. The end of the baffle 1009 facing away from the fixed shaft 1004 and close to the side of the rotating ring 1006 is connected to the inner circumferential surface of the rotating ring 1006. A driving component is arranged outside the airway 10, which is used to drive the rotating ring 1006 to rotate.

The driving component includes: a drive rod 1010 and an electric telescopic rod 1011. One end of the drive rod 1010 passes through the side wall of the airway 10 and connects to the outer wall of the rotating ring 1006. A long slide slot 10012 compatible with the drive rod 1010 is set on the airway 10, and the drive rod 1010 is slidably connected to the long slide slot 10012. The end of the drive rod 1010 facing away from the rotating ring 1006 is equipped with a strip groove 1012. The electric telescopic rod 1011 is arranged on the side wall of the supporting plate 1001, and its output end passes through the strip groove 1012 and is equipped with a connecting rod 1013. One end of the connecting rod 1013 is hingedly connected to the output end of the electric telescopic rod 1011, and the other end of the connecting rod 1013 is hingedly connected to the inner wall of the upper end of the strip groove 1012.

The working principle and beneficial effects of the above technical solution are as follows: Traditional ventilators 2 can adjust the temperature of the respiratory airflow, thus providing patients 6 with suitable respiratory airflow. When compensating the respiratory airflow with carbon dioxide, the temperature of the carbon dioxide airflow being too high or too low can cause significant changes in the temperature of the respiratory airflow, thereby causing damage to the airway and affecting the respiratory experience of the patient 6. Therefore, this solution arranges an electric heating plate 1002 inside the airway 10. The controller 45 can control the electric heating plate 1002 to heat up. When the carbon dioxide airflow passes through the through holes 1003 of the electric heating plate 1002, the passing carbon dioxide airflow can be heated. Setting multiple through holes 1003 can improve the heating efficiency of the carbon dioxide airflow, making the temperature of the carbon dioxide airflow entering the airway 10 consistent with the temperature of the respiratory airflow flowing out of the ventilator 2. The supporting plate 1001 is set on the outer wall of the airway 10, and the supporting plate 1001 can be fixed through bolt installation, thus fixing the airway 10 and improving the stability of the airway 10, avoiding the separation of the airway 10 from the carbon dioxide gas source 1 and affecting the compensation of the carbon dioxide airflow, thereby improving safety and reliability. When the flow rate of carbon dioxide inside the airway 10 needs to be significantly adjusted, the electric telescopic rod 1011 is activated, which then drives the connecting rod 1013 to move. The connecting rod 1013 then drives the drive rod 1010 to move, and the drive rod 1010 drives the rotating ring 1006 to rotate inside the airway 10. As the rotating ring 1006 rotates, it causes one side of the baffle 1009 to deflect, thereby adjusting the angle of the baffle 1009, changing the direction and flow rate of the passing carbon dioxide airflow, and then finely adjusting it through the carbon dioxide regulation proportional valve 444. This not only improves the adjustment precision but also increases the adjustment speed, facilitating rapid adjustments. As the rotating ring 1006 rotates, the second convex ring 1008 and the first convex ring 1007 also rotate relative to each other. The second convex ring 1008 and the first convex ring 1007 cooperate with each other, which can improve the sealing at the connection between the rotating ring 1006 and the fixed ring 1005, avoiding the leakage of carbon dioxide airflow.

### Embodiment 4

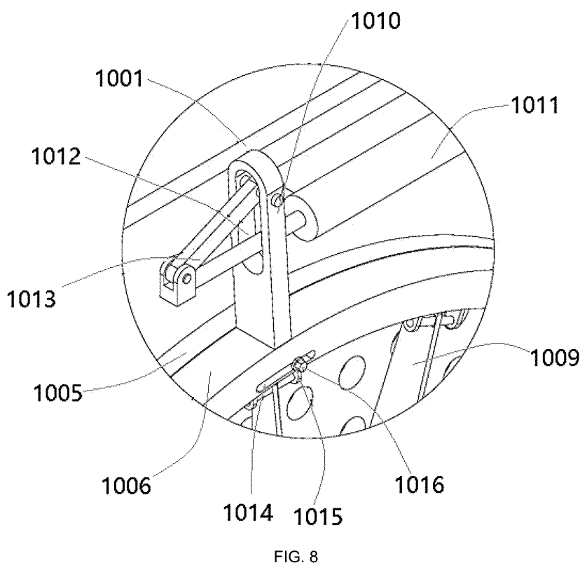

Based on Embodiment 3, as shown in FIG. 8, several arc holes 1014 are opened on the rotating ring 1006, and several arc holes 1014 are arranged in a circular array around the center of the rotating ring 1006. A guide column 1015 is slidably arranged inside the arc hole 1014, one end of the guide column 1015 is fixedly connected to the side of the fixed ring 1005 close to the rotating ring 1006, and the other end of the guide column 1015 extends outside the arc hole 1014 and is equipped with a limit block 1016.

The working principle and beneficial effects of the above technical solution are as follows: The arc hole 1014 is coaxially arranged with the rotating ring 1006. When the rotating ring 1006 rotates, the arc hole 1014 can slide along the guide column 1015, improving the stability of the rotation of the rotating ring 1006. Moreover, the limit block 1016 can limit the rotating ring 1006 to prevent it from separating from the fixed ring 1005, thereby extending the service life of the device.

### Embodiment 5

Based on any one of Embodiments 1-4, as shown in FIG. 1, the disclosure also provides a CO₂ and other gas compensation system connected to a universal ventilator, including the CO₂ and other gas compensation device connected to a universal ventilator mentioned above, as well as the ventilator 2 and the carbon dioxide gas source 1. The carbon dioxide gas source 1 is connected to the input end of the airway 10 of the respiratory gas CO₂ compensation device 9 through the intake pipe 101, and the ventilator 2 is connected to the compensation mixing tube 42 through the respiratory circuit 3.

The working principle and beneficial effects of the above technical solution are as follows: The device body of the disclosure can be connected to all existing universal ventilators 2 on the market, enhancing their functionality and increasing their applicability. The device body can provide ports for supplying carbon dioxide and other active gases to patients 6 for all ventilators 2 on the market, thus expanding the functionality of the ventilator 2, enabling it to provide the required carbon dioxide for patients 6, avoiding respiratory alkalosis in patients 6, greatly improving the therapeutic effect of the ventilator 2, and the device body can adjust the carbon dioxide supply amount and automatically regulate the flow supply according to the changes in airflow parameters such as flow and pressure in the respiratory circuit 3 of the ventilator 2, the proximal pressure, and the end-tidal carbon dioxide value, improving the intelligence and automation level of the system. Moreover, there is no need for communication between the device body and the ventilator 2, which facilitates installation without the need for modifications to the ventilator 2, enhancing its universality.

Obviously, those skilled in the art can make various changes and modifications to the disclosure without departing from the spirit and scope of the disclosure. Thus, if these modifications and variations of the disclosure fall within the scope of the disclosure's claims and their equivalents, the disclosure also intends to include these changes and variations.

What is claimed is:

1. A carbon dioxide (CO₂) and other gas compensation device, configured to be connected to a universal ventilator, wherein the CO₂ and other gas compensation device comprises: a device body, the device body comprises: a respiratory gas CO₂ compensation device, an airway is set inside the respiratory gas CO₂ compensation device, an output end of the airway is connected to a respiratory parameter detection module, the respiratory parameter detection module is connected to a respiratory terminal, a proximal pressure sensor and an end-tidal carbon dioxide sensor are set inside the respiratory terminal, the proximal pressure sensor is used to detect a gas pressure inside the respiratory terminal, the end-tidal carbon dioxide sensor is used to detect an end-tidal CO₂ value inside the respiratory terminal, the proximal pressure sensor and the end-tidal carbon dioxide sensor are electrically connected to the respiratory gas CO₂ compensation device individually, and the respiratory gas CO₂ compensation device is configured to deliver CO₂ to an inside of the respiratory terminal based on detection values of the proximal pressure sensor and the end-tidal carbon dioxide sensor through the respiratory parameter detection module.

2. The CO₂ and other gas compensation device according to claim 1, wherein the respiratory terminal is either a mask or a breathing tube intubation.

3. The CO₂ and other gas compensation device according to claim 1, wherein an intake filter is set near an input end of the airway, and an exhaust filter is set near the output end of the airway.

4. The CO₂ and other gas compensation device according to claim 3, wherein the device body also includes a regulation control system, the regulation control system includes a control unit, an execution unit, and a detection unit, the control unit includes a controller, the controller is set inside the respiratory gas CO₂ compensation device, the controller is electrically connected to the proximal pressure sensor through a first cable, the controller is electrically connected to the end-tidal carbon dioxide sensor through a second cable, the execution unit includes a solenoid valve and a CO₂ regulation proportional valve, both the solenoid valve and the CO₂ regulation proportional valve are set on the airway, the solenoid valve and the CO₂ regulation proportional valve are sequentially set between the intake filter and the exhaust filter, the solenoid valve and the CO₂ regulation proportional valve are electrically connected to the controller individually, the detection unit includes an intake pressure sensor, a CO₂ flowmeter, and a CO₂ supply pressure sensor set in sequence on the airway, the intake pressure sensor, the CO₂ flowmeter, and the CO₂ supply pressure sensor are electrically connected to the controller individually, the intake pressure sensor is located between the intake filter and the solenoid valve, the CO₂ flowmeter is located between the CO₂ regulation proportional valve and the exhaust filter, the CO₂ supply pressure sensor is located between the CO₂ flowmeter and the exhaust filter.

5. The CO₂ and other gas compensation device according to claim 4, wherein the respiratory parameter detection module includes a compensation mixing tube, the compensation mixing tube uses a three-way tube, a first end of the compensation mixing tube is used to introduce airflow from a ventilator, a second end of the compensation mixing tube is connected to the output end of the airway through a connecting tube, a one-way valve is set inside the second end of the compensation mixing tube, a third end of the compensation mixing tube is connected to the inside of the respiratory terminal through a gas supply circuit.

6. The CO₂ and other gas compensation device according to claim 5, wherein the detection unit also includes a flowmeter and a CO₂ concentration sensor, the flowmeter is set near the first end of the compensation mixing tube, the CO₂ concentration sensor is set near the third end of the compensation mixing tube, the flowmeter is electrically connected to the controller through a third cable, and the CO₂ concentration sensor is electrically connected to the controller through a fourth cable.

7. The CO₂ and other gas compensation device according to claim 5, wherein an outside of the airway is equipped with a supporting plate, a plurality of bolt mounting holes are defined on the supporting plate, an inside of the airway is equipped with an electric heating plate, the electric heating plate is located between the intake filter and the intake pressure sensor, the electric heating plate is electrically connected to the controller, the electric heating plate is coaxially set inside the airway, a plurality of through holes are defined on the electric heating plate, a fixed shaft is set in a center of the electric heating plate, a fixed ring and a rotating ring are sequentially set on a side of the electric heating plate near the fixed shaft, an outer circumference of the fixed ring is fixedly connected to an inner wall of the airway, a first convex ring is set on a side of the fixed ring facing away from the electric heating plate, the first convex ring is coaxially set with the fixed ring, an outer circumference of the rotating ring is rotatably connected to the inner wall of the airway, a second convex ring is set on a side of the rotating ring near the fixed ring, an inner side wall of the second convex ring is rotatably connected to an outer side wall of the first convex ring, a plurality of baffles are set on an outer circumference of the fixed shaft, the plurality of baffles are made of a flexible sheet material, the plurality of baffles are arranged in a circular array around the center of the fixed shaft, an end of each baffle is connected to the outer circumference of the fixed shaft, another end of each baffle facing away from the fixed shaft is connected to an inner circumferential surface of the fixed ring on a side of each baffle near the fixed ring, the another end of each baffle facing away from the fixed shaft is connected to an inner circumferential surface of the rotating ring on a side of each baffle near the rotating ring, a driving component is set outside the airway, and the driving component is used to drive the rotating ring to rotate.

8. The CO₂ and other gas compensation device according to claim 7, wherein the driving component includes: a drive rod and an electric telescopic rod, an end of the drive rod penetrates a side wall of the airway and is connected to an outer wall of the rotating ring, a slide slot compatible with the drive rod is defined on the airway, the drive rod is slidably connected to the slide slot, a strip groove is defined on an end of the drive rod facing away from the rotating ring, the electric telescopic rod is set on a side wall of the supporting plate, an output end of the electric telescopic rod passes through the strip groove and is equipped with a connecting rod, an end of the connecting rod is hingedly connected to the output end of the electric telescopic rod, the other end of the connecting rod is hingedly connected to an inner wall of an upper end of the strip groove.

9. The CO₂ and other gas compensation device according to claim 7, wherein a plurality of arc holes are defined on the rotating ring, the plurality of arc holes are arranged in a circular array around a center of the rotating ring, a plurality of guide columns are slidably set inside the plurality of arc holes respectively, an end of each guide column is fixedly connected to a side of the fixed ring near the rotating ring, and the other end of each guide column extends outside the respective arc hole and is equipped with a limit block.

10. A CO₂ and other gas compensation system, comprising: a CO₂ and other gas compensation device, a ventilator and a CO₂ gas source;
wherein the CO₂ and other gas compensation device comprises: a device body, the device body comprises: a respiratory gas CO₂ compensation device, an airway is set inside the respiratory gas CO₂ compensation device, an output end of the airway is connected to a respiratory parameter detection module, the respiratory parameter detection module is connected to a respiratory terminal, a proximal pressure sensor and an end-tidal carbon dioxide sensor are set inside the respiratory terminal, the proximal pressure sensor is used to detect a gas pressure inside the respiratory terminal, the end-tidal carbon dioxide sensor is used to detect an end-tidal CO₂ value inside the respiratory terminal, the proximal pressure sensor and the end-tidal carbon dioxide sensor are electrically connected to the respiratory gas CO₂ compensation device individually, and the respiratory gas CO₂ compensation device is configured to deliver CO₂ to an inside of the respiratory terminal based on detection values of the proximal pressure sensor and the end-tidal carbon dioxide sensor through the respiratory parameter detection module; and
wherein the CO₂ gas source is connected to an input end of the airway through an intake pipe, and the ventilator is connected to the respiratory parameter detection module.

11. The CO₂ and other gas compensation system as claimed in claim 10, wherein the respiratory terminal is a mask.

12. The CO₂ and other gas compensation system as claimed in claim 10, wherein the respiratory terminal is a breathing tube intubation.

13. The CO₂ and other gas compensation system as claimed in claim 10, wherein an intake filter is set near the input end of the airway, and an exhaust filter is set near the output end of the airway.

14. The CO₂ and other gas compensation system as claimed in claim 13, wherein the device body also includes a regulation control system, the regulation control system includes a control unit, an execution unit, and a detection unit, the control unit includes a controller, the controller is set inside the respiratory gas CO₂ compensation device, the controller is electrically connected to the proximal pressure sensor through a first cable, the controller is electrically connected to the end-tidal carbon dioxide sensor through a second cable, the execution unit includes a solenoid valve and a CO₂ regulation proportional valve, both the solenoid valve and the CO₂ regulation proportional valve are set on the airway, the solenoid valve and the CO₂ regulation proportional valve are sequentially set between the intake filter and the exhaust filter, the solenoid valve and the CO₂ regulation proportional valve are electrically connected to the controller individually, the detection unit includes an intake pressure sensor, a CO₂ flowmeter, and a CO₂ supply pressure sensor set in sequence on the airway, the intake pressure sensor, the CO₂ flowmeter, and the CO₂ supply pressure sensor are electrically connected to the controller individually, the intake pressure sensor is located between the intake filter and the solenoid valve, the CO₂ flowmeter is located between the CO₂ regulation proportional valve and the exhaust filter, the CO₂ supply pressure sensor is located between the CO₂ flowmeter and the exhaust filter.

15. The CO₂ and other gas compensation device according to claim 14, wherein the respiratory parameter detection module includes a compensation mixing tube, the compensation mixing tube uses a three-way tube, a first end of the compensation mixing tube is used to introduce airflow from the ventilator, a second end of the compensation mixing tube is connected to the output end of the airway through a connecting tube, a one-way valve is set inside the second end of the compensation mixing tube, a third end of the compensation mixing tube is connected to the inside of the respiratory terminal through a gas supply circuit; and the ventilator is connected to the compensation mixing tube through a respiratory circuit.

16. The CO₂ and other gas compensation device according to claim 15, wherein the detection unit also includes a flowmeter and a CO₂ concentration sensor, the flowmeter is set near the first end of the compensation mixing tube, the CO₂ concentration sensor is set near the third end of the compensation mixing tube, the flowmeter is electrically connected to the controller through a third cable, and the CO₂ concentration sensor is electrically connected to the controller through a fourth cable.

17. The CO₂ and other gas compensation device according to claim 15, wherein an outside of the airway is equipped with a supporting plate, an inside of the airway is equipped with an electric heating plate, the electric heating plate is located between the intake filter and the intake pressure sensor, the electric heating plate is electrically connected to the controller, the electric heating plate is coaxially set inside the airway, a plurality of through holes are defined on the electric heating plate, a fixed shaft is set in a center of the electric heating plate, a fixed ring and a rotating ring are sequentially set on a side of the electric heating plate near the fixed shaft, an outer circumference of the fixed ring is fixedly connected to an inner wall of the airway, a first convex ring is set on a side of the fixed ring facing away from the electric heating plate, the first convex ring is coaxially set with the fixed ring, an outer circumference of the rotating ring is rotatably connected to the inner wall of the airway, a second convex ring is set on a side of the rotating ring near the fixed ring, an inner side wall of the second convex ring is rotatably connected to an outer side wall of the first convex ring, a plurality of baffles are set on an outer circumference of the fixed shaft, the plurality of baffles are made of a flexible sheet material, the plurality of baffles are arranged in a circular array around the center of the fixed shaft, an end of each baffle is connected to the outer circumference of the fixed shaft, another end of each baffle facing away from the fixed shaft is connected to an inner circumferential surface of the fixed ring on a side of each baffle near the fixed ring, the another end of each baffle facing away from the fixed shaft is connected to an inner circumferential surface of the rotating ring on a side of each baffle near the rotating ring, a driving component is set outside the airway, and the driving component is used to drive the rotating ring to rotate.

18. The CO₂ and other gas compensation device according to claim 17, wherein the driving component includes: a drive rod and an electric telescopic rod, an end of the drive rod penetrates a side wall of the airway and is connected to an outer wall of the rotating ring, a strip groove is defined on an end of the drive rod facing away from the rotating ring, the electric telescopic rod is set on a side wall of the supporting plate, an output end of the electric telescopic rod passes through the strip groove and is equipped with a connecting rod, an end of the connecting rod is hingedly connected to the output end of the electric telescopic rod, the other end of the connecting rod is hingedly connected to an inner wall of an upper end of the strip groove.

19. The CO₂ and other gas compensation device according to claim 17, wherein a plurality of arc holes are defined on the rotating ring, the plurality of arc holes are arranged in a circular array around a center of the rotating ring, a plurality of guide columns are slidably set inside the plurality of arc holes respectively, an end of each guide column is fixedly connected to a side of the fixed ring near the rotating ring, and the other end of each guide column extends outside the respective arc hole and is equipped with a limit block.

20. A CO₂ and other gas compensation device, comprising:
a respiratory gas CO₂ compensation device;
an airway, disposed inside the respiratory gas CO₂ compensation device, and configured to be connected with a gas source;
a respiratory parameter detection module, connected to the airway;
a respiratory terminal, connected to the respiratory parameter detection module,
a proximal pressure sensor, disposed inside the respiratory terminal and connected to the respiratory gas CO₂ compensation device, wherein the proximal pressure sensor is configured to detect a gas pressure inside the respiratory terminal;
an end-tidal carbon dioxide sensor, disposed inside the respiratory terminal and connected to the respiratory gas CO₂ compensation device, wherein the end-tidal carbon dioxide sensor is configured to detect an end-tidal CO₂ value inside the respiratory terminal;
wherein the respiratory gas CO₂ compensation device is configured to control the airway to deliver CO₂ from the gas source into the respiratory terminal based on the gas pressure detected by the proximal pressure sensor and the end-tidal CO₂ value detected by the end-tidal carbon dioxide sensor through the respiratory parameter detection module.
